(19) United States
(12) Patent Application Publication
(10) Pub. No.: US 2020/0372999 A1
(43) Pub. Date: Nov. 26, 2020

(54) METHOD FOR CALCULATING FOOD INTAKE AND APPARATUS THEREOF

(71) Applicant: Sang Jun CHOI, Seoul (KR)

(72) Inventor: Sang Jun CHOI, Seoul (KR)

(21) Appl. No.: 16/823,693

(22) Filed: Mar. 19, 2020

(30) Foreign Application Priority Data
May 21, 2019 (KR) 10-2019-0059601

(51) Int. Cl. G16H 20/60; A61B 5/00

(52) U.S. Cl. CPC G16H 20/60; A61B 5/486; A61B 5/742; A61B 5/7405; A61B 5/4205

(57) ABSTRACT

The present embodiment provides a method for calculating food intake comprising, an operation of detecting a biometric reaction of a food intake target; an operation of specifying a biometric trait which identifies the food intake target based on the biometric reaction; an operation of obtaining unit food intake which is an amount of food that the food intake target consumes in one swallow, based on the biometric trait; an operation of measuring the number of swallows of the food intake target; and an operation of calculating total food intake which is a total amount of food consumed by the food intake target from the unit food intake and the number of swallows; wherein the biometric reaction and the unit food intake varies depending on the biometric trait.

### TECHNICAL FIELD

The present embodiment relates to a technique for calculating the food intake of animals or humans.

### BACKGROUND ART

Information of food intake (feed and drink) for pets or livestock is very important. So a breeder can determine the total feed consumed by the livestock by putting in a certain amount of feed in the animal feeder, and subtracting the amount leftover after the livestock is done eating.

However, conventional intake measuring methods are subjective and unscientific. Breeders must remember the amount of feed they initially gave, which is cumbersome and subjective because they must rely on their memory. In addition, in the conventional method, the leftover feed of the livestock is not completely emptied before giving the feed to be eaten for a next meal, but the feed to be eaten for the next meal is added to the feed leftover from the previous meal. Thus, breeders cannot know how much of the feed was consumed by the livestock for each meal. Thus, the conventional method is also unscientific. If several livestock share one feeder or graze, the feed intake of each livestock is difficult to find out.

Meanwhile, it is also necessary for human food intake to be measured. The average healthy person can recognize their food intake. However, if a patient or an elderly person with poor cognition needs to check food intake for health reasons as a dietary need, it can be difficult for them to know exactly what their food intake is.

As such, both humans and animals have a reason to keep track of when and how much they ate. Humans may need food control due to diabetes or obesity, and animals may need health care and food control for breeding purposes. Nevertheless, food intake is often measured depending on the sensory perception.

In this regard, development of a technique for calculating food intake in an objective and rational manner is required.

### PRIOR ART

### Patent Document

(Patent Document 1) Korean Patent Publication No. 2016-0139944, “System for measuring farm feed efficiency and method for measuring farm feed efficiency” (Published on Dec. 7, 2016)

### SUMMARY OF THE INVENTION

### Technical Problem

In such a background, the present embodiment is directed to provide a food intake calculation technique that calculates the total amount of food consumed in each meal from biometric reactions (vibration or sound coming from the throat) from the swallowing of humans and animals.

### Technical Solution

In order to achieve the above object, an embodiment provides a method for calculating food intake performed by a food intake calculating apparatus which includes, an operation of detecting a biometric reaction of a food intake target; an operation of specifying a biometric trait which identifies the food intake target based on the biometric reaction; an operation of obtaining unit food intake which is an amount of food that the food intake target consumes in one swallow, based on the biometric trait; an operation of measuring the number of swallows of the food intake target; and an operation of calculating total food intake which is a total amount of food consumed by the food intake target from the unit food intake and the number of swallows; wherein the biometric reaction and the unit food intake varies depending on the biometric trait.

In the above method, the biometric reaction may include at least one of a vibration, a sound, a radio frequency, and light, from a body of an animal or human, and the biometric trait may include at least one of a type and a size of the food intake target.

In the above method, an operation of storing a result where the biometric reaction and the biometric trait corresponding to the biometric reaction are mutually matched, may be included.

In the above method, the biometric reaction may include at least one of a vibration, a sound, a radio frequency, and light, from a body of an animal or human, and the biometric trait may include at least one of a type and a size of the food intake target, and the result may be from mutually matching the at least one of a vibration, a sound, a radio frequency, and light, and the at least one of a type and a size, and the operation of specifying may specify the at least one of a type and a size based on the at least one of a vibration, a sound, a radio frequency, and light.

In the above method, an operation of storing a unit food intake corresponding to the biometric trait may be included, and the operation of obtaining may read a unit food intake corresponding to the specified biometric trait from the stored unit food intake.

In the above method, the biometric trait may include at least one of a type and a size of the food intake target, and the unit food intake may be stored corresponding to the at least one of a type and a size, and the operation of obtaining may read a unit food intake that corresponds to the specified at least one of a type and a size from the stored unit food intake.

In the above method, the operation of measuring may measure the number of swallows from at least one of a vibration, a sound, a radio frequency, and light of the biometric reaction and may enter an active state for measuring upon detecting the biometric reaction.

Another embodiment provides, a food intake calculating apparatus including a sensor unit that detects the biometric reaction for a food intake target; and a control unit that specifies a biometric trait which identifies the food intake target based on the biometric reaction, and obtains unit food intake which is an amount of food that the food intake target consumes in one swallow, based on the biometric trait; wherein the control unit measures the number of swallows of the food intake target, and calculates total food intake which is a total amount of food consumed by the food intake target from the unit food intake and the number of swallows, wherein the biometric reaction and the unit food intake varies depending on the biometric trait.

In the above apparatus, an output unit that visually or audibly outputs the total food intake may be included.

In the above apparatus, a storage unit that stores a result where the biometric reaction and a biometric trait corresponding to the biometric reaction are mutually matched, and stores a unit food intake corresponding to the biometric trait may be included, wherein the biometric reaction may include at least one of a vibration, a sound, a radio frequency, and light, from a body of an animal or human, and wherein the biometric trait may include at least one of a type and a size of the food intake target, wherein the control unit may specify the at least one of a type and a size based on the at least one of a vibration, a sound, a radio frequency, and light, and may read a unit food intake corresponding to the specified at least one of a type and a size from the stored unit food intake.

### Advantageous Effects

As described above, according to the present embodiment, it is possible to calculate the total amount of food consumed in each meal from the biometric reaction from the swallowing of humans or animals. Accordingly, the calculation of the total food intake can be objective and rational without depending on the sense of the measurer.

The total food intake can be calculated differently depending on the type and physical characteristics of the food intake target. Accordingly, it is possible for the calculation of the total food intake to be adaptively changed according to the type and physical characteristics of the food intake target.

### BRIEF DESCRIPTION OF THE DRAWINGS

FIG. 1 is a conceptual view illustrating a mode in which a food intake calculating apparatus is used according to an embodiment.

FIG. 2 is a configuration diagram illustrating a food intake calculating apparatus according to an embodiment.

FIG. 3 is an exemplary view illustrating a method of operating a food intake calculating apparatus according to an embodiment.

FIG. 4 is an exemplary view illustrating that data of a biometric reaction and a biometric trait are mutually matched and stored in a table form according to an embodiment.

FIG. 5 is an exemplary view illustrating that data of a biometric trait and a unit food intake are mutually matched and stored in a table form according to an embodiment.

FIG. 6 is a flowchart illustrating an operation of the food intake calculating apparatus according to an embodiment.

FIG. 7 is a flowchart illustrating an operation of each component of the food intake calculating apparatus according to an embodiment.

FIG. 8 is a flowchart illustrating an operation of each component of the food intake calculating apparatus according to another embodiment.

FIG. 9 is a conceptual diagram illustrating a mode in which a food intake calculating system is used according to yet another embodiment.

FIG. 10 is a configuration diagram illustrating a food intake calculating system according to yet another embodiment.

### DETAILED DESCRIPTION OF THE INVENTION

Hereinafter, some embodiments of the present disclosure will be described in detail with reference to the accompanying drawings. In adding reference numerals to the components of each drawing, it should be noted that the same components have the same reference numerals as much as possible, even if they appear on other figures. In addition, when the present disclosure is described and a detailed description of a related known configuration or function is determined to obscure the gist of the present disclosure, a detailed description thereof will be omitted.

Further, in describing the components of the present disclosure, terms such as first, second, A, B, (a), and (b) can be used. These terms are merely used to distinguish the components from other components, and the terms do not limit the essence, order, or order of the components. If any component is described as “joined,” “coupled,” or “connected” to another component, that component may be directly joined or connected to the other component, but it is to be understood that another component may be “joined,” “coupled,” or “connected” between each component.

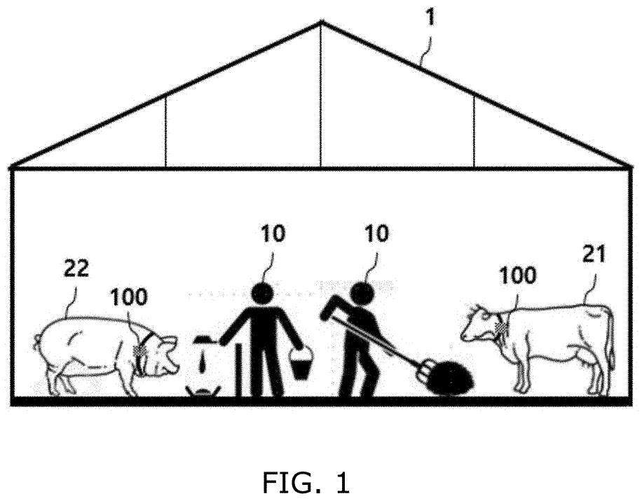

FIG. 1 is a conceptual view illustrating a mode in which a food intake calculating apparatus is used according to an embodiment.

Referring to FIG. 1, a breeder 10 can know the amount of food consumed by livestock 21, 22 via a food intake calculating apparatus 100.

In a breeding farm 1 where livestock 21, 22 stays, the breeder 10 feeds the livestock 21, 22 with feed and drink every meal, and the livestock 21, 22 eats the feed and drink. Livestock 21, 22 may generally mean a cow 21, a pig 22 and/or poultry, but may contain primarily other herbivores with grass as a main diet. The breeding farm 1 can include stables, barns and pigsties as places where livestock 21, 22 is fed. The breeder 10 can be a person who manages the livestock 21, 22 and who has a need to know how much food the livestock 21, 22 consumes.

Meanwhile, the food intake calculating apparatus 100 can calculate the amount of food that the livestock 21, 22 eats. The amount of food eaten by livestock 21, 22 can be termed food intake. The food intake calculating apparatus 100 detects a specific biometric reaction coming out of the livestock 21, 22, and determines whether the livestock 21, 22 is passing food to the esophagus and can measure the amount of consumed food, from the detected biometric reaction.

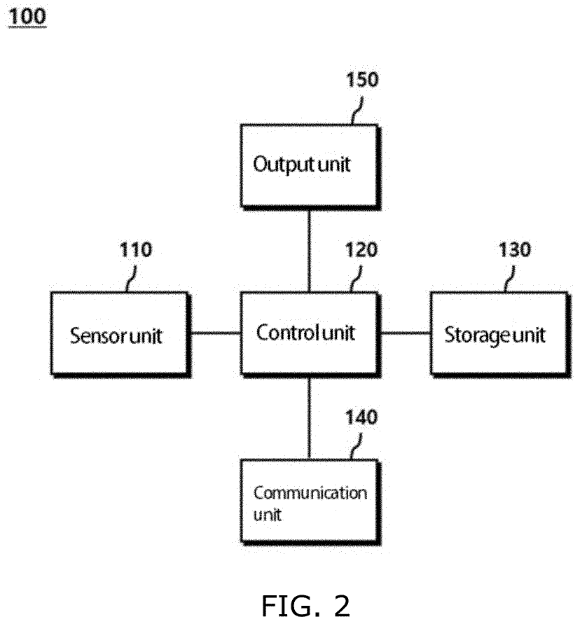

FIG. 2 is a configuration diagram illustrating a food intake calculating apparatus according to an embodiment.

Referring to FIG. 2, each configuration of the food intake calculating apparatus 100 according to an embodiment is shown. The food intake calculating apparatus 100 may include a sensor unit 110, a control unit 120, a storage unit 130, a communication unit 140, and an output unit 150.

The sensor unit 110 can detect a biometric reaction of a food intake target. The food intake target may indicate an animal having a structure that mainly swallows food through the esophagus. The above food intake target may include not only animals but also humans. As long as the purpose of using the food intake calculating apparatus 100 is to measure food intake, livestock 21, 22 or patients can mainly applicable as the food intake target.

The sensor unit 110, among biometric reactions, can detect a wave (vibration or sound) generated when the food intake target passes the food down its throat. Therefore, the sensor unit 110 may include an acceleration sensor, a gyro sensor, an infrared sensor, a microphone, a radio frequency detector, a camera, or the like. Since it is necessary for the sensor unit 110 to detect a biometric reaction that occurs when the food intake target swallows, the sensor unit 110 may be located on a body part where the neck or esophagus of the food intake target is located. The sensor unit 110 can better detect the vibration and/or sound of the above-mentioned swallowing from the part where the neck or esophagus is located than other parts of the body. However, the sensor unit 110 is not limited to being located on the neck and the esophagus, and may be located in other body parts.

The reason why the sensor unit 110 detects the swallowing vibration or sound (the wave from the throat) of the food intake target as a biometric reaction is because the swallowing vibration or sound varies depending on the type (species) or physical characteristics (height, weight or esophagus diameter) of the food intake target.

The above-mentioned swallowing vibrations and sounds are composed of a series of wavelengths, each of which can be combined (nested or offset) with a unique frequency and amplitude to form a unique wavelength. The unique wavelength (above-mentioned swallowing vibrations and sounds) may vary depending on the type (species) or physical characteristics of the food intake target. For example, the vibration and sound wavelength of a swallow of a horse, a cow 21, and a pig 22 are different from that of a human, and the vibration or sound of a swallow of a same cow 21 may vary depending on weight. Therefore, when the wavelength of the above-mentioned swallowing vibration or sound is analyzed, it is possible to figure out the type and physical characteristics of the food intake target making the above-mentioned swallowing vibration or sound.

The sensor unit 110, among the biometric reactions, can detect a wave (sound or vibration) generated when the food intake target chews food or drinks (before swallowing). Since it is necessary for the sensor unit 110 to detect a biometric reaction that occurs when the food intake target chews food or drinks, the sensor unit 110 may be located at a body part where the mouth of the food intake target is located. The sensor unit 110 can detect the vibration and/or sound of the swallowing in the part where the mouth is located, more than in other parts of the body. However, the sensor unit 110 is not limited to the mouth and can be located in other body parts.

The sensor unit 110 can detect, in addition to vibration and sound, a wave including radio frequency and light emitted from the food intake target. For example, body temperature can increase when livestock 21, 22 or humans chews or swallows food. The change of body temperature can be displayed by infrared rays, and the sensor unit 110 can detect the infrared rays. Also, when livestock 21, 22 or a person chews or swallows food, a specific radio frequency (brain wave) can be generated by the brain, and the sensor unit 110 can detect the brain wave. The sensor unit 110 can detect a radio frequency or light in a specific band emitted from the body.

As described above, the sensor unit 110 can detect a medium containing a wave from livestock 21, 22 or a human. For example, the sensor unit 110 can detect sound, vibration, light, radio frequencies, or the like. Waves detected from sound, vibration, light or radio frequency are recognized as a biometric reaction by the control unit 120, and may be used to calculate the food type and total food intake and specify the biometric trait. Hereinafter, sound and vibration will be described as examples, but this description can also be applied to light and radio frequency.

The control unit 120 can specify a biometric trait for identifying the food intake target based on a biometric reaction. Each of the biometric reactions can be different depending on the biometric traits. For example, the swallowing vibration and sound of a cow 21 may be different from the swallowing vibration and sound of a pig 22. In addition, vibration and sound may vary depending on the weight even among cows 21. This is because each of the wavelengths that make up the vibration and sound are different. Therefore, the control unit 120 can derive a specific biometric trait using the wavelength of the detected biometric reaction. For example, the control unit 120 may analyze a wavelength pattern of the detected biometric reaction, and from among biometric trait data stored in the storage unit 130, may read biometric trait data matched with a wavelength pattern having the same wavelength pattern as the analyzed wavelength pattern.

The biometric trait may indicate a biological property for identifying the above-mentioned food intake target. For example, biometric traits may include the type (species) and physical characteristics (height, weight, or esophagus diameter) of the food intake target. The physical characteristics may be a concept that includes the body size or largeness of the food intake target such as height, weight or esophagus diameter. The type of food intake target can indicate whether the food intake target is human or animal, and of the animals, whether the animal is a horse, cow 21 or pig 22. The physical characteristics of the food intake target can indicate at least one of height, weight, and esophagus diameter of a human or animal.

The control unit 120 may obtain a unit food intake based on the biometric traits. The unit food intake may indicate the amount of food consumed in one swallow by the food intake target. The unit food intake may vary depending on the biometric traits. For example, the unit food intake can be different depending on the type of food intake target. The unit food intake may vary depending on the food intake target and whether it is a human or animal, and may vary depending on whether the animal is a horse, cow 21 or pig 22. As another example, unit food intake may also vary depending on physical characteristics. Unit food intake may vary depending on height, weight or esophagus diameter. This is because the unit food intake can be increased or decreased in proportion to the height, weight or esophagus diameter.

In order to obtain the unit food intake, the control 120 may read the unit food intake data matched with a biometric trait that is the same as the specified biometric trait, from unit food intake data stored in the storage unit 130. For example, when the above-mentioned specified biometric trait indicates a person weighing 70 kg, the control unit 120 reads out the unit food intake data corresponding to the person weighing 70 kg from the storage unit 130. Thereby, the unit food intake can be obtained.

The control unit 120 can measure the number of swallows of food intake target. The control unit 120 can measure the number of swallows from the biometric reaction detected by the sensor unit 110.

For example, the control unit 120 can analyze the vibration or sound wave of the swallowing among the biometric reactions and measure the number of swallows. As one method, the control unit 120 may determine that swallowing has occurred each time a specific wave pattern is repeated in the biometric reaction, and may increment (count-up) the number of swallows by one. As another method, the control unit 120 may determine that a swallowing has occurred each time a pause period between the repetition of the specific pattern appears, and may increase the number of swallows by one. As another method, the control unit 120 may increase the number of swallows by one, for each time a specific pattern appears that is generated in a biometric reaction when swallowing occurs.

When the control unit 120 detects a biometric reaction from the food intake target, the control unit 120 can enter an active state for measuring the number of swallows. The control unit 120 can start measuring the number of swallows only after entering the active state. For example, in the above-described example of measuring the number of swallows, the control unit 120 enters the active state at the moment when the vibration or sound of the swallowing is detected by the sensor unit 110, and then when any one of the repetition of the specific wave pattern from the inside, the rest period between the repetition of the wave patterns, and the specific wave pattern, is recognized, the first count can be started.

The control unit 120 can enter an inactive state in which the measurement of the number of swallows is ended when no biometric reaction is detected from the food intake target. When the control unit 120 enters the inactive state, it can end the measurement of the number of swallows. For example, if vibration or sound of the swallowing are no longer detected by the sensor unit 110 and the control unit 120 has not received any wavelength data from the sensor unit 110 for a certain period of time, the control unit 120 can enter the inactive state and end the measurement of the number of swallows.

When the control unit 120 detects an unusual biometric reaction from the food intake target, the control unit 120 can enter an inactive state in which the measurement of the number of swallows is ended. When the control unit 120 enters the inactive state, it can end the measurement of the number of swallows. For example, when the sensor 110 detects the vibration or sound of a burp that the livestock 21, 22 lets out after swallowing, and the control unit 120 receives data of a specific wavelength from the sensor unit 110, the control unit 120 can enter the inactive state and end the measurement of the number of swallows.

The control unit 120 can record the starting time and ending time of food consumption of the food intake target. The control unit 120 can regard the time of entering the active state as the time of starting food consumption, that is, the start of each meal. The control unit 120 can record the time at which the active state is entered in the storage unit 130. On the other hand, the control unit 120 can regard the point in time of entering the inactive state as the ending time of food consumption, that is, the end of each meal. The control unit 120 can record the time at which the inactive state is entered in the storage unit 130.

The control unit 120 can record in the storage unit 130 the time the food intake target starts and ends food consumption, together with the total food intake. In the example described above, the control unit 120 can record the time of entering the active state and the time of entering the inactive state in the storage unit 130 together with the calculated total food intake.

The control unit 120 can calculate a total food intake that is a total amount of food consumed by the food intake target. For example, the control unit 120 can calculate the total food intake from the unit food intake obtained based on the biometric traits and the measured number of swallows. Since the unit food intake means food intake per swallow, the control unit 120 can calculate the multiplication of the measured number of swallows by the unit food intake as the total food intake.

The storage unit 130 may store data necessary for calculating the total food intake. The storage unit 130 may store data on biometric reactions, biometric traits, and unit food intake. In the storage unit 130, the biometric reaction and the corresponding biometric trait can be matched and stored. In addition, the biometric traits and the corresponding unit food intake can be matched and stored in the storage unit 130. The control unit 120 can read a part of the biometric trait data in the storage unit 130 based on the detected biometric reaction. The control unit 120 can read a part of the unit food intake data of the storage unit 130 based on the specified biometric trait.

The communication unit 140 can transmit or receive data. The communication unit 140 can receive the data for calculating the total food intake from the control unit 922, and can transmit the data back to an external device (not shown). The external device may output the data of the calculation of the total food intake visually or audibly. The breeder 10 can visually or audibly recognize the total food intake of the livestock 21, 22 of each meal via the external device.

The output unit 150 can receive and output the data of the calculation of the total food intake from the control unit 120. For example, the food intake calculating device 100 may include a display as an output unit 150, and the breeder 10 can visually recognize the total food intake of the livestock 21, 22 of each meal via the output unit 150. In addition, the food intake calculating device 100 can include a speaker as an output unit 150, and the breeder 10 can audibly recognize the total food intake of the livestock 21, 22 of each meal via the output unit 150.

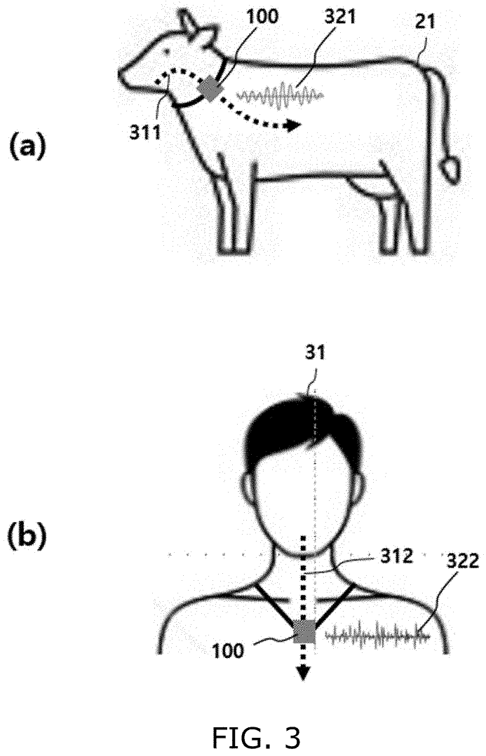

FIG. 3 is an exemplary view illustrating a method of operating a food intake calculating apparatus according to an embodiment.

Referring to FIG. 3, the food intake calculating apparatus 100 may be coupled to a person or animal and be operated. In particular, the sensor unit 110 of the food intake calculating apparatus 100 can be coupled so as to be as close as possible to a position where a biometric reaction occurs in a human or animal body. This is because the sensor unit 110 can clearly detect the biometric reaction signal only when doing so. However, the sensor unit 110 does not necessarily have to be close to a position where a biometric reaction occurs, and can be coupled to a position far away from this.

FIG. 3a can illustrate that the food intake calculating apparatus 100 is coupled to the neck of a cow 21 among the animals. The food intake calculating apparatus 100 can detect the vibration signal 321 emitted when the cow 21 eats the feed. As the feed proceeds along the esophagus 311 (arrow) of the cow, the sensor unit 110 can detect a vibration signal 321. The sensor unit 110 for detecting the vibration signal 321 may be arranged as close as possible to the neck of the cow 21 or its periphery.

FIG. 3b can illustrate that a food intake calculating apparatus 100 is coupled to the neck of a person, in particular, a patient 31. The food intake calculating apparatus 100 can detect a sound signal 322 emitted when the patient 31 eats food. As the food proceeds along the patient's esophagus 312 (arrow), the sensor unit 110 can detect the audio signal 322. The sensor unit 110 for detecting the sound signal 322 may be arranged as close as possible to the neck of the patient 31 or its periphery.

As mentioned above, the food intake calculating apparatus 100 is preferably located at or near the source of the signal (throat or esophagus). However, when the detection sensitivity of the signal of the sensor unit 110 is excellent, the food intake calculating apparatus 100 including the sensor unit 110 may also be located in other body parts far away from the source. For example, the food intake calculating apparatus 100 may be located on a leg or hip part of the cow 21 and can detect a vibration signal 321 emitted from the esophagus.

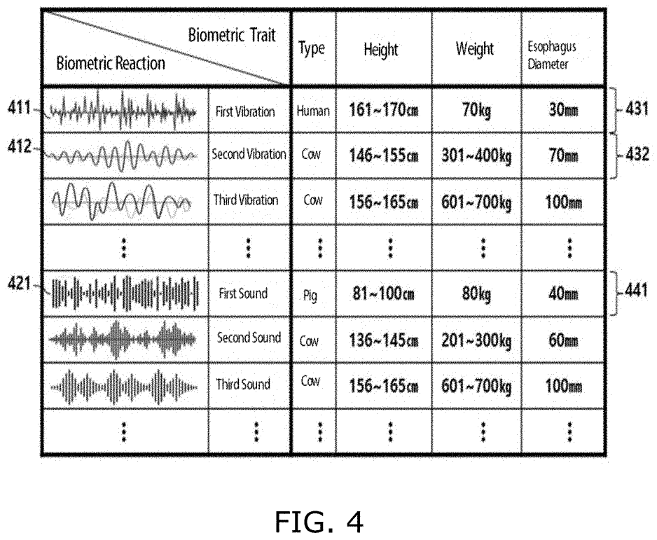

FIG. 4 is an exemplary view illustrating that data of a biometric reaction and a biometric trait are mutually matched and stored in a table form according to an embodiment.

Referring to FIG. 4, a graphical representation of the data stored in the storage 130 is shown. The biometric reaction data and the biometric trait data can be stored in the storage unit 130 in correspondence with each other. The correspondence can be represented by graphically representing the biometric reaction data and the biometric trait data in the form of a table.

The biometric reaction can be matched one-on-one with the biometric traits. If the biometric reaction is vibration and the biometric traits are type, height, weight and esophagus diameter, the waveform of the vibration may correspond to type, height, weight and esophagus diameter with a unique value or range. Other biometric reactions may include sound, radio frequencies and light. Hereinafter, vibration and sound will be described as examples, but this description can also be applied to a case where light or radio frequencies are used as a biometric reaction.

For example, of the biometric reaction, the first vibration can have a first vibration waveform 411. The first vibration and the first vibration waveform 411 may correspond to the first vibration biometric trait 431. The first vibration biometric trait 431 may indicate that the type of the food intake target is a human, the height is 161-170 cm, the weight is 70 kg, and the esophagus diameter is 30 mm. The first vibration and data in a state where the first vibration waveform 411 is matched with the first vibration biometric trait 431 may be stored in the storage unit 130. If the control unit 120 determines that the biometric reaction detected by the sensor unit 110 is the first vibration waveform 411, the control unit 120 may read the first vibration biometric trait 431. The control unit 120 can identify the food intake target as a person having a height of 161-170 cm, a weight of 70 kg, and an esophagus diameter of 30 mm. The control unit 120 can identify the food intake target by specifying the first vibration biometric trait 431 based on the first vibration. The control unit 120 can obtain the unit food intake based on the identified first vibration biometric trait 431.

Biometric reactions and biometric traits may be for animals. For example, of the biometric reaction, the second vibration may have a second vibration waveform 412. The second vibration and the second vibration waveform 412 may correspond to the second vibration biometric trait 432. The second vibration biometric trait 432 may indicate that the type of food intake target is a cow, the height is 146-155 cm, the weight is 301-400 kg, and the esophagus diameter is 70 mm. The second vibration and data in a state where the second vibration waveform 412 is matched with the second vibration biometric trait 432 may be stored in the storage unit 130. When the control unit 120 determines that the biometric reaction detected by the sensor unit 110 is the second vibration waveform 412, the control unit 120 may read the second vibration biometric trait 432. The control unit 120 may identify the food intake target as a cow having a height of 146-155 cm, a weight of 301-400 kg, and an esophagus diameter of 70 mm. The control unit 120 may identify the food intake target by specifying the second vibration biometric trait 432 based on the second vibration. The control unit 120 can obtain the unit food intake based on the identified second vibration biometric trait 432.

Biometric reactions may be about sound. For example, among the biometric reactions, the first sound may have a first sound waveform 421. The first sound and the first sound waveform 421 may correspond to the first sound biometric trait 441. The first sound biometric trait 441 may indicate that the type of food intake target is a pig, its height is 81-100 cm, its weight is 80 kg, and its esophagus diameter is 40 mm. The first sound and data in a state where the first sound waveform 421 are matched with the first sound biometric trait 441 may be stored in the storage unit 130. If the control unit 120 determines that the biometric reaction detected by the sensor unit 110 is the first sound waveform 421, the control unit 120 may read the first sound biometric trait 441. The control unit 120 can identify the food intake target as a pig having a height of 81-100 cm, a weight of 80 kg and an esophagus diameter of 40 mm. The control unit 120 may specify the first sound biometric trait 441 based on the first sound and identify the food intake target. The control unit 120 can obtain the unit food intake based on the identified first sound biometric trait 441.

The biometric reaction data and the biometric trait data may be numerical values predetermined through experiments. Biometric reactions may vary depending on the biometric traits. Vibrations or sounds from swallowing may vary depending on the type of food intake target, the weight, height, or the esophagus diameter. For example, a person having a height of 161-170 cm, a weight of 70 kg, and an esophagus diameter of 30 mm may emit a first vibration having a first vibration waveform 411 during food consumption. In addition, a cow having a height of 146-155 cm, a weight of 301-400 kg, and an esophagus diameter of 70 mm may emit a second vibration having a second vibration waveform 421 at the time of feeding. Such correspondence can be confirmed through several experiments on humans and animals. This result may be stored in a storage unit 130 in the form of a database.

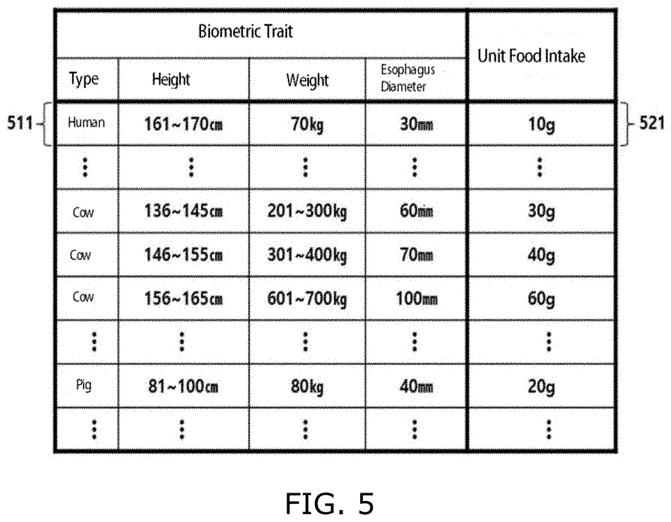

FIG. 5 is an exemplary view illustrating that data of a biometric trait and a unit food intake are mutually matched and stored in a table form according to an embodiment.

Referring to FIG. 5, a graphical representation of the data stored in the storage unit 130 is shown. The biometric trait data and the data on the amount of intake of the unit food may be stored in the storage unit 130 in correspondence with each other. The correspondence relationship can be represented by graphically expressing the biometric trait data and the data on the unit food intake in the form of a table.

The biometric trait may be matched one-on-one with the unit food intake. Biometric traits, including at least one of type, height, weight, and esophagus diameter, can correspond to unit food intake of the amount of food that passes through the esophagus during one swallow.

For example, the first biometric feature 511 may indicate that the type of food intake target is a person, whose height is 161-170 cm, weight is 70 kg, and esophagus diameter is 30 mm. The first biometric trait 511 may correspond to the first unit food intake 521. The first unit food intake 521 may indicate that 10 g of food is consumed in a single swallow. Data in a state where the first biometric trait 511 and the first unit food intake 521 are matched may be stored in the storage unit 130. If the control unit 120 determines that the food intake target has the first biometric trait, the control unit 120 may read the first unit food intake 521. The control unit 120 can obtain the unit food intake of a person having a height of 161-170 cm, a weight of 70 kg, and an esophagus diameter of 30 mm. The control unit 120 may calculate the total food intake based on the obtained first unit food intake 521.

On the other hand, the control unit 120 may obtain the unit food intake by using only some of the specific biometric traits from the biometric reaction. For example, when the control unit 120 specifies the first vibration biometric trait 431 as the biometric trait of the food intake target from the first vibration, it is possible to read a first unit food intake which corresponds to a weight of 70 kg among the first vibration biometric trait 431. The control unit 120 can obtain a unit food intake of 10 g of the food intake target.

The biometric trait data and the data on the unit food intake may be predetermined through experiments. The unit food intake may vary depending on the biometric traits. The amount of food consumed in a single swallow may vary depending on the type of food intake target, the weight, height or esophagus diameter. For example, a person whose height is 161-170 cm, weight is 70 kg, and has an esophagus diameter of 30 mm may consume about 10 g per swallow. In addition, a cow having height of 146-155 cm, weight of 301-400 kg, and an esophagus diameter of 70 mm may consume about 40 g per swallow. Such correspondence can be confirmed through several experiments on humans and animals. This result may be stored in a storage unit 130 in the form of a database.

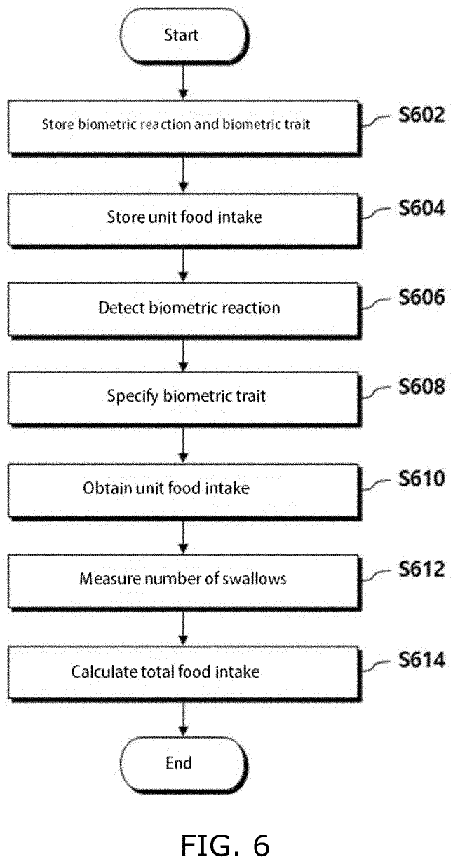

FIG. 6 is a flowchart illustrating an operation of the food intake calculating apparatus according to an embodiment.

Referring to FIG. 6, there is shown an operation order of the food intake calculating apparatus 100 according to the present disclosure.

The food intake calculating apparatus 100 may receive data on biometric reactions and biometric traits and store them in the storage unit 130 in step S602. The data of the biometric reaction and the biometric traits may be stored in the form of a table in which biometric reactions corresponding to the biometric traits are mutually matched. The correspondence may be predetermined through experiments on humans and animals.

The food intake calculating apparatus 100 may receive data on the unit food intake and store it in the storage unit 130 in step S604. The data on the unit food intake may be stored in the form of a table in which the unit food intakes corresponding to the biometric traits are mutually matched. The correspondence may be predetermined through experiments on humans and animals.

The food intake calculating apparatus 100 may detect a biometric reaction via the sensor unit 110 in step S606. The biometric reaction may include at least one of a vibration and a sound generated when passing through the esophagus.

The food intake calculating apparatus 100 may specify the biometric traits via the control unit 120 in step S608. The food intake calculating apparatus 100 may specify the biometric trait corresponding to the biometric reaction by reading from the storage unit 130. The biometric traits may include at least one of species (type), height, weight, and esophagus diameter as physical traits of the food intake target.

The food intake calculating apparatus 100 may obtain the unit food intake via the control unit 120 in step S610. The food intake calculating apparatus 100 may obtain the unit food intake corresponding to the biometric traits by reading the unit food intake from the storage unit 130.

The food intake calculating apparatus 100 may measure the number of swallows of the food intake target via the control unit 120 in step S612. The food intake calculating apparatus 100 may measure the number of swallows from the biometric reaction. The food intake calculating apparatus 100 may measure the number of swallows by analyzing the waveform of vibration or sound emitted from the swallowing.

The food intake calculating apparatus 100 may calculate the total food intake via the control unit 120 in step S614. The food intake calculating apparatus 100 may calculate the total food intake by multiplying the obtained unit food intake by the measured number of swallows.

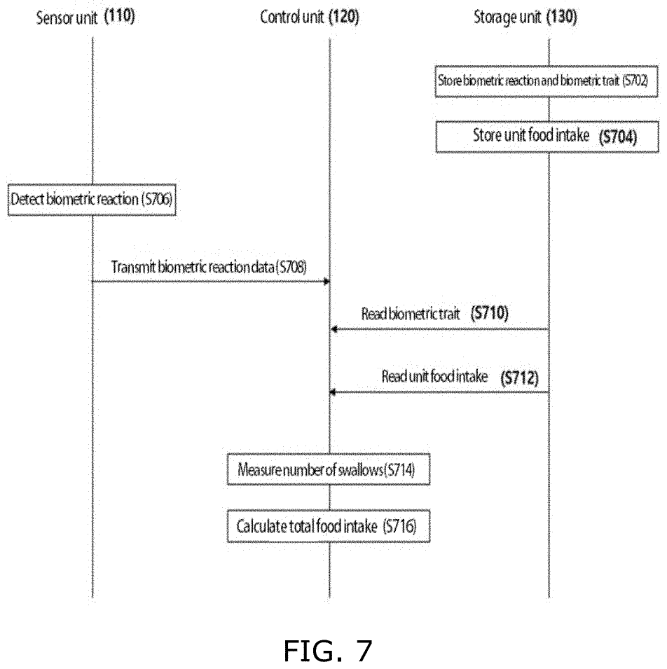

FIG. 7 is a flowchart illustrating an operation of each component of the food intake calculating apparatus according to an embodiment.

Referring to FIG. 7, the operating order of each of the components of the food intake calculating apparatus 100 according to the present disclosure is shown.

The storage unit 130 may store data of a biometric reaction and biometric traits corresponding to the biometric reaction in step S702. Then, the storage unit 130 may store data of unit food intake corresponding to the biometric traits in step S704.

The sensor unit 110 may detect a biometric reaction by being attached to a human or animal body in step S706. The sensor unit 110 may detect vibrations or sounds emitted from the throat and its periphery.

The sensor unit 110 may transmit data on the detected biometric reaction to the control unit 120 in step S708.

The control unit 120 may specify biometric traits. The control unit 120 may read the specified biometric trait corresponding to the detected biometric reaction from the storage unit 130 in step S710.

The control unit 120 may obtain the unit food intake. The control unit 120 may read the unit food intake corresponding to the specified biometric trait from the storage unit 130 in step S712.

The control unit 120 may measure the number of swallows of the food intake target in step S714. The control unit 120 may derive the number of swallows from the detected biometric reaction. The control unit 120 may perform a pattern analysis of the waveform of the vibration or sound emitted from the swallowing.

The control unit 120 may calculate the total food intake based on the read unit food intake and the measured number of swallows in step S716.

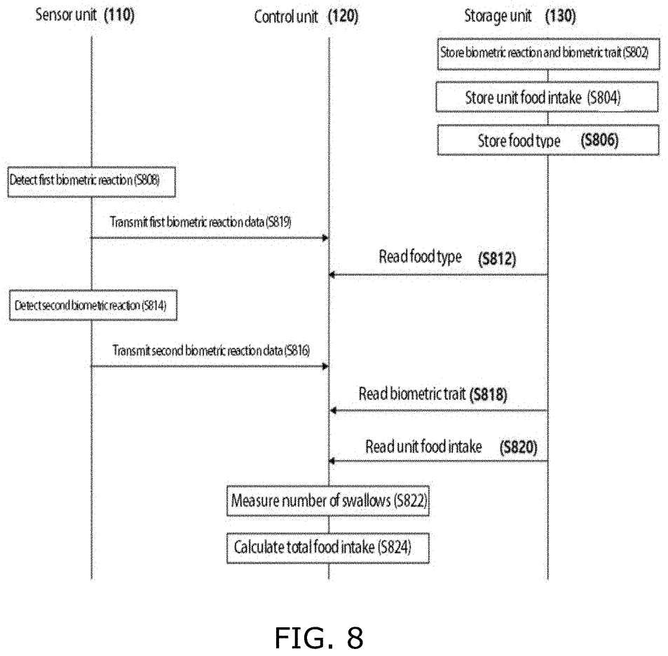

FIG. 8 is a flowchart illustrating an operation of each component of the food intake calculating apparatus according to another embodiment.

Referring to FIG. 8, the operating order of each of the components of the food intake calculating apparatus 100 according to the present disclosure is shown. Unlike FIG. 7, the food intake calculating apparatus 100 may identify the type of food consumed by the food intake target from a biometric reaction. For example, the food intake calculating apparatus 100 can identify whether the livestock 21, 22 is eating feed or drinking water.

In the storage unit 130, the biometric reaction and the data on corresponding biometric traits may be stored in step S802. The storage unit 130 may store data on the unit food intake corresponding to the biometric traits in step S804. Then, the storage unit 130 may store data of the type of food corresponding to the biometric reaction in step S806. The data of the type of food corresponding to the biometric reaction may include a result of matching of a specific food type according to the specific biometric reaction. The data of the type of food corresponding to the biometric reaction may be stored in the storage unit 130 in a table form. For example, the biometric reaction 1A can be matched with the feed A, the biometric reaction 1B can be matched with the feed B, and the biometric reaction 2A can be matched with the drink A and stored in the storage unit 130.

The sensor unit 110 may be attached to a human or animal body, and may detect a first biometric reaction in step S808. The sensor unit 110 may detect at least one of a vibration, a sound, a radio frequency, and light from the mouth and its periphery as the first biometric reaction. Hereinafter, sound and vibration will be described as examples.

The sensor unit 110 may detect a different biometric reaction according to the type of food that the food intake target consumes. For example, if a cow 21 consumes feed or water, it may chew the feed (food chewing exercise) before swallowing or drink water. Here, the sounds and vibrations emitted when chewing may be different from the sounds and vibrations emitted when drinking. The sensor unit 110 may detect another sound or vibration that comes out when chewing or drinking through the mouth. The sensor unit 110 may detect a sound or a vibration that occurs when chewing feed before swallowing or drinking water as the first biometric reaction.

Further, the sensor unit 110 may subdivide and detect the type of food. For example, in the example described above, let's assume that cow 21 consumes feed A and feed B. The sound and vibration emitted when cow 21 chews feed A and the sound and vibration emitted when chewing feed B may differ. The sensor unit 110 may detect sounds or vibrations generated when the feed A or the feed B is chewed in the mouth, respectively. The sensor unit 110 may detect, as the first biometric reaction, a sound or a vibration generated when the feed A or the feed B is chewed before swallowing.

In another example, in the example described above, let's assume that cow 21 consumes feed A and drink A. The sound or vibrations emitted when cow 21 chews feed A and the sounds or vibrations emitted when drinking a drink A may differ. The sensor unit 110 may detect sounds or vibrations generated when the feed A is chewed in the mouth and sounds or vibrations generated when the drink A is drunk. The sensor unit 110 may detect a sound or vibration made when the feed A is chewed before swallowing as a first biometric reaction. In addition, the sensor unit 110 may detect a sound or vibration generated when the drink A is drunk before swallowing as the first biometric reaction.

The sensor unit 110 may transmit the data on the detected first biometric reaction to the control unit 120 in step S810.

The control unit 120 may specify the type of food. The control unit 120 may read the data of the type of food corresponding to the detected first biometric reaction from the storage unit 130 in step S812. For example, in the above example, if the first biometric reaction corresponds to biometric reaction 1A, the data of feed A matched with biometric reaction 1A may be read from storage unit 130. The control unit 120 may specify the food currently consumed by the cow 21 as the feed A from the first biometric reaction. In addition, if the first biometric reaction corresponds to biometric reaction 1B, the data of feed B matched with biometric reaction 1B may be read from storage unit 130. The control unit 120 may specify the food currently consumed by the cow 21 as the feed B from the first biometric reaction. If the first biometric reaction corresponds to the biometric reaction 2A, the control 120 may read the data of the drink A matched with the biometric reaction 2A from the storage unit 130. The control unit 120 may specify the food currently consumed by the cow 21 as the drink A from the first biometric reaction.

The sensor unit 110 may be attached to a human or animal body, and may detect a second biometric reaction in step S814. The sensor unit 110 may detect a vibration or a sound emitted from the throat or its periphery as the second biometric reaction.

The sensor unit 110 may transmit the data of the detected second biometric reaction to the control unit 120 in step S816.

The control unit 120 may specify biometric traits. The control unit 120 may read the biometric trait corresponding to the detected second biometric reaction from the storage unit 130 in step S818.

The control unit 120 may obtain the unit food intake. The control unit 120 may read the unit food intake corresponding to the specific biometric trait from the storage unit 130 in step S820.

The control unit 120 may measure the number of swallows of the food intake target in step S822. The control unit 120 may derive the number of swallows from the detected second biometric reaction. The control unit may perform a pattern analysis of the waveform of the vibration or sound emitted from the swallowing.

The control unit 120 may calculate the total food intake based on the read unit food intake and the measured number of swallows in step S824.

According to the steps described above, the food intake calculating apparatus 100 may calculate different total food intakes according to the type of food. When the control unit 120 of the food intake calculating apparatus 100 specifies the type of food from the first biometric reaction and calculates the total food intake from the second biometric reaction that subsequently appears, the total food intake consumed by the food intake target can be derived for each food.

For example, when the cow 21 eats the feed A, the control unit 120 may specify that the feed A is being consumed, from the first biometric reaction (biometric reaction 1A) that comes out when the cow 21 chews the feed A (during mastication). Subsequently, the control unit 120 may calculate the total food intake of the feed A from the second biometric reaction that occurs when the cow 21 passes the feed A down its throat. Therefore, the user 10 can know how much of the feed A the cow 21 ate via the output unit 150. When the cow 21 drinks the drink A, the control unit 120 may specify that the cow 21 is drinking (sucking) the drink A from the first biometric reaction (biometric reaction 2A) that occurs when the cow 21 drinks the drink A. Subsequently, the control unit 120 may calculate the total food intake of the drink A from the second biometric reaction that occurs when the cow 21 passes the drink A down its throat. Therefore, the user 10 can know how much of the drink A the cow 21 drank via the output unit 150.

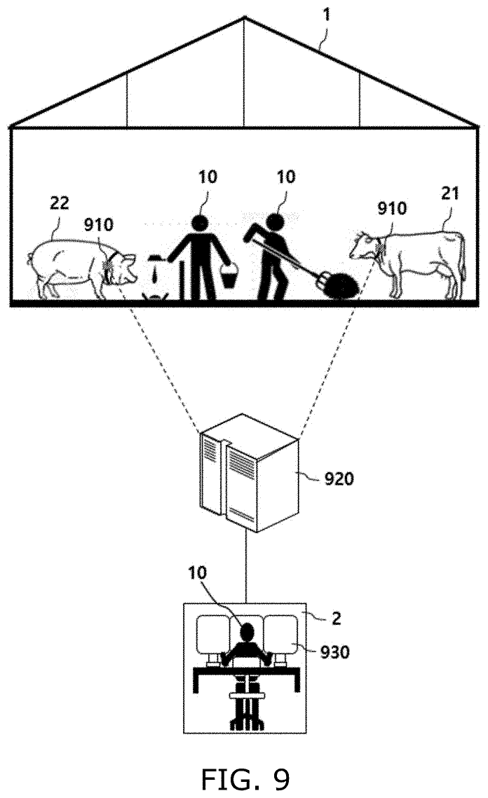

FIG. 9 is a conceptual diagram illustrating a mode in which a food intake calculating system is used according to yet another embodiment.

Referring to FIG. 9, a breeder 10 can remotely know the amount of food consumed by livestock 21, 22 via a food intake calculation system 800 according to another embodiment.

The detection of the biometric reaction and the output calculation of the total food intake may be performed in another place. In FIG. 1, a food intake calculating apparatus 100 according to an embodiment may be characterized in that both a sensor unit 110 and a control unit 120 are coupled to livestock 21, 22 inside a breeding farm 1. Therefore, the breeder 10 can know the total food intake only by looking at the food intake calculating apparatus 100 attached to the livestock 21, 22.

On the other hand, in FIG. 9, a food intake calculation system 900 according to another embodiment includes a sensor device 910 for detecting a biometric signal, which is coupled to livestock 21, 22, and a server 920 for executing the calculation of the total food intake, which is located outside the breeding farm 1.

The server 920 may receive the detected biometric reaction data from the sensor communication unit 912 via an internal server communication unit 921. The server 920 may calculate the total food intake via a control unit 922. The server 920 may transmit the calculation result of the total food intake to a user terminal 930 via the server communication unit 921. The user terminal 930 may display the calculation result to the breeder 10 via an output unit 932. The breeder 10 can know the total food intake via the output unit 931 of the user terminal 930 in a management office 2. The breeder 10 can remotely know the total food intake of the livestock 21, 22 without going directly to the breeding farm 1.

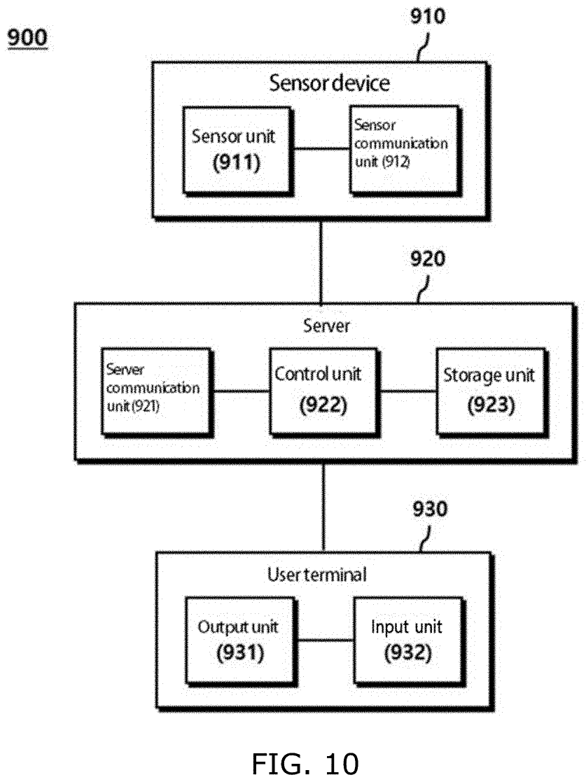

FIG. 10 is a configuration diagram illustrating a food intake calculating system according to yet another embodiment.

Referring to FIG. 10, each configuration of a food intake calculating system 900 according to another embodiment is shown. The food intake calculating system 900 may include a sensor device 910, a server 920 and a user terminal 930. The sensor device 910 may include a sensor unit 911 and a sensor communication unit 912. The server 920 may include a server communication unit 921, a control unit 922, and a storage unit 923. The user terminal 930 may include an output unit 931 and an input unit 932.

The sensor unit 911 of the sensor device 910 may perform the same function as the sensor unit 110 of the food intake calculating apparatus 100. The sensor unit 911 may detect a biometric signal. The sensor unit 911 may deliver the detected biometric signal to the sensor communication unit 912.

The sensor communication unit 912 of the sensor device 910 may transmit or receive data. The sensor communication unit 912 may receive biometric reaction data from the sensor unit 911. The biometric reaction data relates to vibration or sound, and may include a unique wave. The sensor communication unit 912 may transmit the received biometric reaction data to the server communication unit 921 of the server 920.

The server communication unit 921 of the server 920 may transmit or receive data. The server communication unit 921 may receive the biometric reaction data from the sensor communication unit 912 and deliver it to the control unit 922.

The control unit 922 of the server 920 may perform the same function as the control unit 120 of the food intake calculating apparatus 100. The control unit 922 may specify a biometric trait from the biometric reaction data, obtain an intake amount of the unit food based on the specific biometric trait, measure the number of swallows, and determine the intake amount of the unit food. The total food intake may be calculated based on the number of swallows.

The storage unit 923 of the server 920 may perform the same function as the storage unit 130 of the food intake calculating apparatus 100. The storage unit 923 may store data of a result obtained by matching a biometric reaction and a biometric trait in a table form. The storage unit 923 may store data of a result obtained by matching the biometric traits and the intake amount of the unit food in a table form.

The server communication unit 921 may transmit data on the total food intake calculated by the control unit 922 to the output unit 931 of the user terminal 930.

The output unit 931 of the user terminal 930 may output the total food intake calculated by the control unit 922. The output unit 931 may receive and output the data for the calculation of the total food intake from the control unit 922. For example, the user terminal 930 may be a remote device such as a mobile device or a PC, and the output unit 931 may be a display or a monitor. The breeder 10 can know the total food intake of the livestock 21, 22 of each meal through the output unit 931.

The input unit 932 of the user terminal 930 may input the data required for calculating the total food intake. The input unit 932 may input at least one of biometric reaction data, biometric trait data, unit food intake data, and food type data. The input unit 932 may transmit the at least one biometric reaction data, the biometric trait data, the unit food intake data and the food type data to the server 920. The data received from the input unit 932 may be stored in the storage unit 923 of the server 920.

Terms such as “including”, “constituting” or “having”, described above, mean that the component can be inherent unless stated otherwise, so it should be construed that the components are not excluded but rather may further include other components. Unless defined otherwise, all terms, including technical or scientific terms, have the same meaning as commonly understood by one of ordinary skill in the art. Commonly used terms, such as pre-defined terms, should be construed to be consistent with the contextual meaning of the relevant art and should not be interpreted as an ideal or excessively formal meaning, unless explicitly defined herein.

The above description is merely illustrative of the technical concept of the present disclosure, and for any person having ordinary knowledge in the art, various modifications and variations can be made within a range that does not depart from the essential characteristics of the present disclosure. Therefore, the embodiments disclosed herein are not intended to limit the technical idea of the present disclosure but to explain the present disclosure, and the scope of the technical idea of the present disclosure is not limited by such embodiments. The protected scope of the present disclosure should be construed according to the following claims, and all technical ideas falling within the scope of equivalents should be construed as being included in the scope of the present disclosure.

1. A method for calculating food intake performed by a food intake calculating apparatus, the method comprising,
an operation of detecting a biometric reaction of a food intake target;
an operation of specifying a biometric trait which identifies the food intake target based on the biometric reaction;
an operation of obtaining unit food intake which is an amount of food that the food intake target consumes in one swallow, based on the biometric trait;
an operation of measuring the number of swallows of the food intake target; and
an operation of calculating total food intake which is a total amount of food consumed by the food intake target from the unit food intake and the number of swallows;
further comprising an operation of storing a result where the biometric reaction and the biometric trait corresponding to the biometric reaction are mutually matched; and an operation of storing a result where the biometric trait and the unit food intake corresponding to the biometric trait are mutually matched,
wherein the operation of specifying analyzes a pattern of the biometric reaction and reads a biometric trait matched with the pattern of the biometric reaction,
wherein the operation of obtaining reads a unit food intake matched with the read biometric trait,
wherein the biometric trait is specified by the biometric reaction,
and wherein the unit food intake is obtained by the biometric trait.

2. The method of claim 1,
wherein the biometric reaction comprises at least one of a vibration, a sound, a radio frequency, and light, from a body of an animal or human,
and wherein the biometric trait comprises at least one of a type and a size of the food intake target.

3. The method of claim 1,
wherein the operation of calculating calculates the total food intake by multiplying the unit food intake and the number of swallows.

4. The method of claim 1,
wherein the biometric reaction comprises at least one of a vibration, a sound, a radio frequency, and light, from a body of an animal or human,
wherein the biometric trait comprises at least one of a type and a size of the food intake target,
wherein the result where the biometric reaction and the biometric trait corresponding to the biometric reaction are mutually matched, is from mutually matching the at least one of a vibration, a sound, a radio frequency, and light, and the at least one of a type and a size,
wherein the operation of specifying specifies the at least one of a type and a size based on the at least one of a vibration, a sound, a radio frequency, and light.

5. The method of claim 1,
wherein the operation of measuring determines that a swallow occurs every time a specific form of the pattern is generated or every time a specific form of the pattern is repeated, and counts the number of swallows.

6. The method of claim 1,
wherein the biometric trait comprises at least one of a type and a size of the food intake target, and
wherein the unit food intake is stored corresponding to the at least one of a type and a size,
wherein the operation of obtaining reads a unit food intake that corresponds to the specified at least one of a type and a size from the stored unit food intake.

7. The method of claim 1,
wherein the operation of measuring measures the number of swallows from at least one of a vibration, a sound, a radio frequency, and light of the biometric reaction and enters an active state for measuring upon detecting the biometric reaction.

8. A food intake calculating apparatus comprising,
a storage unit that stores a result where a biometric reaction and a biometric trait corresponding to the biometric reaction are mutually matched, and a result where the biometric trait and a unit food intake corresponding to the biometric trait are mutually matched;
a sensor unit that detects the biometric reaction for a food intake target; and
a control unit that specifies a biometric trait which identifies the food intake target based on the biometric reaction, obtains unit food intake which is an amount of food that the food intake target consumes in one swallow, based on the biometric trait, measures the number of swallows of the food intake target, and calculates total food intake which is a total amount of food consumed by the food intake target from the unit food intake and the number of swallows,
wherein the control unit analyzes a pattern of the biometric reaction and reads a biometric trait matched with the pattern of the biometric reaction in order to specify the biometric trait, and reads a unit food intake matched with the read biometric trait in order to obtain the unit food intake,
wherein the biometric trait is specified by the biometric reaction,
and wherein the unit food intake is obtained by the biometric trait.

9. The apparatus of claim 8, comprising an output unit that visually or audibly outputs the total food intake.

10. The apparatus of claim 8,
wherein the biometric reaction comprises at least one of a vibration, a sound, a radio frequency, and light, from a body of an animal or human,
wherein the biometric trait comprises at least one of a type and a size of the food intake target,
wherein the control unit specifies the at least one of a type and a size based on the at least one of a vibration, a sound, a radio frequency, and light, and reads a unit food intake corresponding to the specified at least one of a type and a size from the stored unit food intake.
